(19) United States
(12) Patent Application Publication
(10) Pub. No.: US 2015/0065813 A1
(43) Pub. Date: Mar. 5, 2015

(54) SYSTEM FOR RECORDING AND PROCESSING SIGNAL FOR DIAGNOSING AUDITORY SYSTEM AND METHOD FOR RECORDING AND PROCESSING SIGNAL FOR DIAGNOSING AUDITORY SYSTEM

(71) Applicant: SILVERMEDIA Spolka z ograniczona odpowiedzialnoscia, Krakow (PL)

(72) Inventors: Ireneusz Wochlik, Jaroslaw (PL); Jaroslaw Bulka, Krakow (PL); Michal Front, Rudawa (PL); Piotr Pracuch, Krakow (PL); Lukasz Folwarczny, Jaroslaw (PL)

(73) Assignee: SILVERMEDIA Spolka z ograniczona odpowiedzialnoscia, Krakow (PL)

(21) Appl. No.: 14/042,639

(22) Filed: Sep. 30, 2013

(30) Foreign Application Priority Data
Sep. 3, 2013 (PL) P.405230

(51) Int. Cl. A61B 5/0484; A61B 5/12

(52) U.S. Cl. CPC A61B 5/04845; A61B 5/125
USPC 600/301; 600/544; 600/546; 600/559

(57) ABSTRACT

In system for recording and processing a signal for diagnosing an auditory system, a system for measuring signals of bioelectric activity of brain (EEG signal) or eye (EOG signal) or muscle (EMG signal) or their combination obtained from electrodes (11, 21, 22) attached to head skin of a person to be tested communicates with system (105) informing about sleep phase occurrence through an input (27). A system for measuring an auditory brainstem response signal (ABR signal) evoked by acoustic stimulation is automatically activated by a circuit (106) for automatic activation connected to the system (105) informing about sleep phase occurrence and measures the ABR signal. The system also includes a recording system (101, 102) of the ABR signal in a configurable period of time measured from the time of acoustic stimulation and system (103) to present the ABR signal in graphical form.

### CROSS-REFERENCE TO RELATED APPLICATIONS

Pursuant to 35 U.S.C. 119 and the Pans Convention Treaty this application claims the benefit of Polish Patent Application No. P.405230 filed on Sep. 3, 2013, the contents of which are incorporated herein by reference.

### BACKGROUND OF THE INVENTION

1. Field of the Invention

Technical concept presented herein relates to a system for recording and processing a signal for diagnosing an auditory system. This relates further to a method for recording and processing a signal for diagnosing an auditory system.

2. Description of the Related Art

Increasing urbanization means that more and more people are being affected by sounds of increasing intensity, which sometimes results in problems with the auditory system, especially for people working in companies where noise is generated with a high intensity. Early detection of problems with the auditory system could lead to initiation of treatment leading to a result that at worst is to not make hearing problems more and more severe.

The most common method of diagnosing auditory system is to generate certain sounds, which are then repeated by the tested person. The degree of accuracy in imitating generated sound proves the efficiency of the auditory system.

From the description JPH08266518 A titled ‘Earphone unit for audition test device’ is known a headset for a hearing testing device, which is very convenient to use for the person conducting the test and for the one being tested. This headset is designed in such a way that the data cable is connected to the device testing hearing and a switch response element that generates a response signal is connected to its ending. A switch response element is used to send out an answer to the main unit operated by the person carrying out the test depending if the patient can hear the test tone or not.

Another known method for diagnosing the auditory system is the electrophysiological audiometry method, which is based on indirect electric bio-potential registration, resulting from the activation of the following sections of the auditory pathway of the nervous system caused by acoustic stimulus. The registration is performed using techniques derived from the EEG, and the secondary potentials are registered and can be measured on the surface of the head skin. The study called Auditory Evoked Potentials (AEP) is the registration of electrical activity that can be observed on the surface of the skull. During the study electrical activity is processed in a manner which gives information about the brain's response to acoustic stimuli and suggest characteristics relevant to the research project.

Encephalogram (EEG) techniques and Auditory Brainstem Evoked Response (ABR) research referring to methods of diagnosing the auditory system are known from publication titled ‘Human Sleep and Sleep EEG’, written by K. \check{S}u\check{s}máková, publication titled ‘Bone-Conduction ABR: Clinically Feasible And Clinically Valuable’, written by James W. Hall III, and publication titled ‘Pediatric Auditory Brainstem Response Assessment: The Cross-Check Principle Twenty Years Later’, written by Katheryn Rupp Bachmann and James W. Hall III.

From the publication US2011224569 A1 ‘Method and device for removing EEG artifacts’ are known systems and methods for automatically identifying signal segments, EEG signal or other activity of the brain which contain artifacts. After the identification of such segments, they can be edited in order to remove them from the signal.

### SUMMARY OF THE INVENTION

It is an object of the present invention to provide a system for recording and processing with a simple and easy to use design for the diagnosis of the auditory system. It would divide signal for the diagnosis of the auditory system separating noise and clutters, and would present a signal to the auditory system diagnostics for simple signal analysis in the diagnosis of the auditory system.

This objective is achieved by a system for recording and processing of the signals for the auditory system diagnostics, including electrodes connected to the head skin and near the auditory system of the tested person, a system for acoustic stimulation of the auditory system, systems for measuring bioelectric signals of brain or eye activity, and muscle twitching activity: electroencephalogram (EEG) or electrooculogram (EOG) or electromyogram (EMG) and auditory brainstem evoked response signal (ABR signal) caused by acoustic stimulation, amplifiers and filters, systems for recording EEG or EOG or EMG or their combination, and also an ABR signal in a timeline, a system for analysis and processing of EEG or EOG or EMG or their combination and the ABR signal to obtain information concerning the response of the brain to an acoustic stimulus and to remove artifacts caused by the movement of the electrodes or wires and muscle artifacts and systems that aim to present EEG or EOG or EMG or their combination, and ABR signal in the form of charts, characterized by a system of measuring the bioelectric activity of brain signals or eye EEG or EOG or EMG or their combination, consisting of an independent measurement channel EEG or EOG or EMG or their combination of the electrodes connected on the head skin of a tested object, communicating with the system informing the phase of sleep by observing input, generating the activation signal and having the output of the signal of activation and the system for measuring the signal induced by stimulation of the acoustic ABR has an automatic activation system of an independent ABR channel with the input signal activating communication with the output of the activated signal of the information system which recorded a phase of sleep and the independent channel for ABR signal acquisition with electrodes attached near the auditory system of the tested object, which is activated by the input line after receiving the activation signal from the output of the automatic activation of the track ABR, amplifiers amplifying ABR signal up to 500 mV, obtained from an independent channel for ABR signal acquisition and limiting the way of transmitting by using fillers in the range 0 Hz to 5000 Hz, the signal converter circuit independent channel for ABR signal acquisition from analog to digital recording system, a system recording a signal of an independent ABR path signal acquisition in a configurable period of time measured from the acoustic stimulation and signal system to provide an independent channel for ABR signal acquisition in graphical form.

Independent measurement channel EEG or EOG or EMG, or their combinations, and the independent channel for ABR signal acquisition can be connected via the processor through the communications interface circuits for external communication together with a transmitting device and a receiving and data processing unit of measurement data processing which is carried out by an independent measurement channel EEG or EOG or EMG or their combinations, and the independent channel for ABR signal acquisition.

The object of the present invention is to also provide a method of recording and signal processing for the diagnosis of the auditory system that would allow the separation of the signal for the diagnosis of the auditory system from noise and clutters in a short period of time, and present it in graphical form.

The method of recording and processing signal for the diagnosis of the auditory system, according to which after connecting the electrodes on the head skin and near the auditory system, data of bioelectric signals of brain activity or eye or muscle twitching: EEG or EOG or EMG or their combinations, are recorded, and the signal evoked by the auditory brainstem response signal (ABR) caused by acoustic stimulation of the auditory system, which is processed in graphic form in order to obtain information on the brain's response to acoustic stimulation, removing artifacts caused by the movement of electrodes or wires and muscle artifacts. The recording shows its course over time in a graph, characterized in that the first charge signal is bioelectrical activity of the brain or eye or muscle through an independent measuring channel of EEG or EOG or EMG or their combination from the electrodes connected on the head skin, which is converted from analog to digital, and after observing the optimal baseline ABR sleep phase signals, generated acoustic stimulation of the auditory system by acoustic stimulation circuit and activating signal generated by the system informing to start observing the optimal sleep phase ABR testing, which shall be automatic activation of an independent channel for ABR signal acquisition. Later it automatically activates independent ABR circuit signal acquisition and signal ABR is collected by electrodes placed in the vicinity of the auditory system and independent channel for ABR signal acquisition, which amplifies the amplifier to a voltage not higher than 500 mV, and is limited to a range of transfer from 0 Hz to 5000 Hz by means of filters, and then performs a signal conversion of ABR signal of independent channel of ABR signal acquisition from analog to digital and records ABR signal of independent channel for ABR signal acquisition in a configurable time interval measured from the acoustic stimulation, and ABR signal of independent channel for ABR signal acquisition are presented in graphical form on a screen or on paper.

Preferably, the signal of bioelectric activity of the brain or eyes, obtained by an independent measurement channel EEG or EOG or EMG or their combination from the electrodes on the head skin and an independent channel of ABR signal, after the conversion from analog to digital is sent through the interface communication systems for external communication together with the transmitting device to the receiving and processing unit of the data from the measurements made by an independent measurement channel EEG or EOG or EMG, or their combination, and the independent channel for ABR signal acquisition.

By using an independent measurement channel EEG or EOG or EMG, or their combination, and the independent channel for ABR signal acquisition, running after the detection of sleep phase, the time for recording and processing of data for the diagnosis of the auditory system has been significantly shortened and simplified.

### BRIEF DESCRIPTION OF THE DRAWINGS

This and other objects as well as advantageous features of the technical concept presented herein are accomplished in accordance with the principles of the presented technical concept by providing a system for recording and processing a signal for diagnoses of an auditory system. Further details and features of the system and method, its nature and various advantages will become more apparent from the accompanying drawings and the following detailed description of the preferred embodiments shown in a drawing, in which:

FIG. 1 shows a record of idealized auditory brain stem response potentials;

FIG. 2 shows a system for registering and processing signals for diagnosis of an auditory system:

FIG. 3 shows a system for recording and processing signals for diagnosis of the auditory system conducted remotely;

FIG. 4 shows a block diagram of an example ABR device;

FIG. 5 shows schematically the amplitude spectrum and the ABR signal;

FIG. 6 shows a block diagram of algorithm of the defection of sleep phase;

FIG. 7 shows a block diagram of algorithm of the detection ABR signal;

FIGS. 8A, 8B and 8C show a block diagram of a system for wave detection;

FIGS. 9A, 9B, 9C and 9D show a block diagram illustrating processes of communication with the ABR device with control application;

FIGS. 10A and 10B show a block diagram of algorithm of sleep phase detection;

FIG. 11 illustrates the waveform EEG or EOG or EMG or their combination with a part showing the presence of sleep phase 2, 3 or 4;

FIGS. 12A and 12B show a block diagram of recording signals for diagnosis of the auditory system by means of ABR device;

FIG. 13 shows a ABR signal waveform;

FIGS. 14A, 14B and 14C show a block diagram of the extraction of ABR signal; and

FIG. 15 shows a sat of graphs of ABR signals of a properly hearing person with decreasing intensity of the acoustic signal.

### DETAILED DESCRIPTION OF EMBODIMENTS

The embodiments presented in the drawings are intended only for illustrative purpose and do not limit the scope of the present invention, as defined by the accompanying claims.

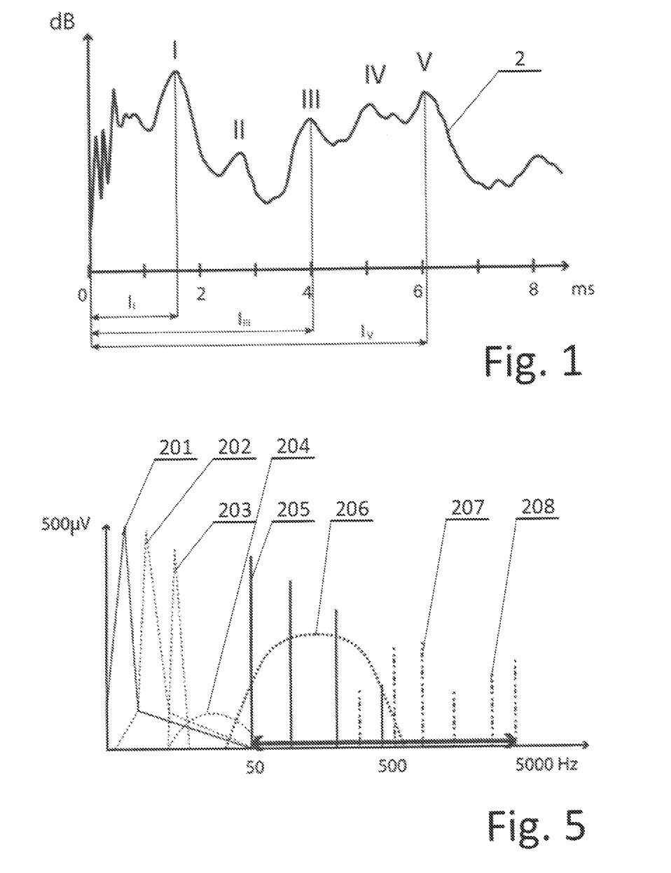

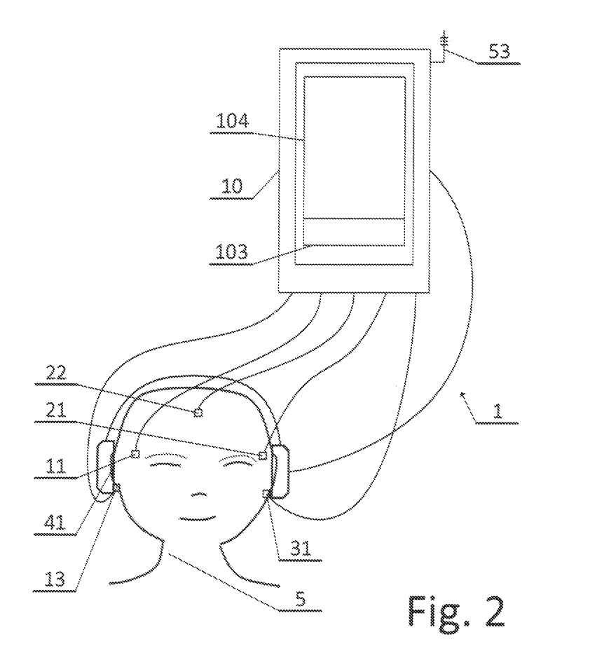

A system for recording and processing a signal for diagnosing an auditory system, shown in FIGS. 2, 3 and 4, as well as a method for recording and processing the signal for diagnosing the auditory system described below, refers to auditory evoked potential, presented as bioelectric activity of the brain caused by acoustic stimulation. Part of this potential, which includes the response time of acoustic stimulus of about 10 ms after the stimulus, is called Auditory Brainstem Response (ABR) signal or fast potential. Auditory Brainstem Response signal hereinafter called the ASR signal 2, as shown in FIG. 1, is notable by a few characteristic peaks, which are called waves, marked by Roman numerals I-VII, whose search is based on the morphological analysis of the recorded signals and analysis of the latencies and amplitudes of individual waves as a function of the intensity of the stimulation signal, of which in FIG. 1 have been marked only waves I-V. In the illustrated examples of the invention a crack or tone is used as a stimulating signal whose intensity is reduced gradually during the test run. For each current ABR waveform, the ABR signal is obtained, where the above waves I-V and interval I_{I}, I_{III} and I_{V} can be selected. In the ABR signal recording, there are three to five predominant peaks, which depend on the magnitude and type of stimulus, age, sex and type of hearing loss. The threshold of hearing corresponds to such noise levels at which the ABR signal recording can still distinct the structure of the corresponding wave V, while a further weakening of the signal causes the complete disappearance of wave V. In the presented examples of the invention, evoked potentials are recorded almost exclusively by means of surface electrodes, which are at different distances from the various structures of the auditory system involved in the generation of individual potentials, for example, located on the mastoid process or earlobes. In this situation electrodes predominantly receive bioelectric activity of cerebral cortex while brainstem activity or cochlea that are further away from the electrodes attenuate in spontaneous bioelectrical activity of the cerebral cortex.

As mentioned earlier, the auditory system is more than a dozen different potentials generated in the cochlea, the auditory nerve, brain stem, and subcortical centers of the codex. These potentials are distinguished by the time between giving impetus to the emergence of the individual peaks of the waves. This time is referred to as latency. Latency value is in the range from a few to a few hundred milliseconds, and is dependent on the area of the auditory system from which the potentials originate from. There are three types of potentials, namely short latency responses (SLR), where the time between giving impetus to its emergence ranges up to 10 ms, potentials middle latency responses (MLR), generated in the range of 10-100 ms, and the long latency responses (LLR), generated in more than 100 ms. Short latency responses potentials contain a series of several waves, and their appearance is attributed to the activation of the classical auditory nuclei of the auditory pathway in the brainstem, including the cochlear dorsal, ventral and olivary nuclei, and nuclei of lateral lemniscus. Potentials of the medium and long latency are caused during the activation of the cortex, resulting in less clarity and greater variability between individuals.

Recordings of auditory brain stem evoked potentials, in accordance with the invention, are recorded in a specified number of milliseconds after the stimulus, e.g. 10 ms, after the acoustic stimulus. Idealized response consisting of several waves indicated in FIG. 1, in accordance with the accepted nomenclature of Roman numerals,
Wave I is generated in the distal part of the auditory nerve;
Wave II is generated in the proximal part;
Wave III is generated in the anterior cochlear nucleus and dorsal cochlear nucleus;
Wave IV is generated mainly in the nucleus olivari superior;
Wave V is generated mainly in nuclei of lateral lemniscus.

The characteristics of the correct answer in the form of ABR waveform, which should be logged by the registration system and signal processing for the diagnosis of the auditory system are:

normal morphology, in the case where the waves I, III, V are visible in the recording having preserved ratios between the amplitudes of each wave, where the amplitude of the wave V is the highest, the amplitude of the third wave is somewhat less, and the smallest wave amplitude is wave I;

a highly rate of repeatability of the answer while keeping parameters of stimulation the same;

the correct value intervals and latency;

the correct ratio between intervals I-III and III-V, and despite the fact that the intervals may be in the standard, the system must enable verification of whether interval I-III is slightly longer than interval III-V.

Any pathology within the anatomical structures related to the auditory system may change the morphology of recording and time parameters, which means latency, intervals and also amplitudes of particular waves. In the case of serious pathology that leads to neurogenic deafness, the course of a signal recorded may completely lack relevant structures.

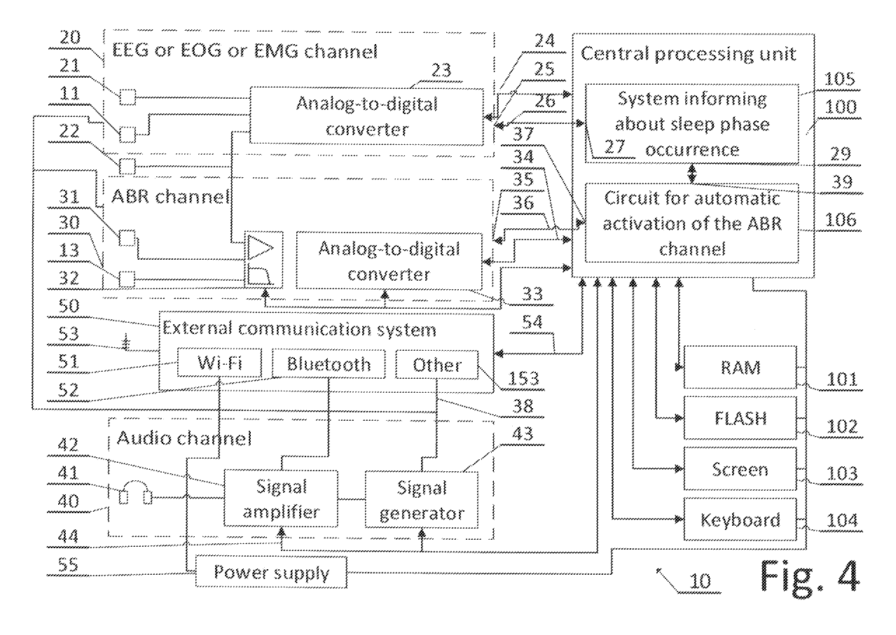

The system 1 of measuring, downloading and recording signals for diagnosis of the auditory system, abbreviated the system 1, shown in FIG. 2, allows the conduction of objective examinations of hearing using auditory brainstem response method (ASR), even in places where the tested object is located. The system 1 comprises an ABR device 10 placed in a package that contains the system for measuring signals of bioelectric activity of brain, eyes or muscles EEG or EOG or EMG or their combination with independent channel for signals acquisition EEG or EOG or EMG or their combination, as shown in FIG. 4, with electrodes 11, 21, 22 attached to the skin of the head of tested object 5, namely a person to be tested, communicating with system informing about sleep phase occurrence, shown in FIG. 4, generating activation signal and having activation signal output, which may be a separate system or part of the processor controlling the whole system 1.

The ABR 10 device, which is a part of system 1, also contains, as shown in FIG. 4, a system for measuring ABR signal induced by acoustic stimulation with independent channel of ABR signal acquisition, shown in FIG. 4, with electrodes 13 and 31 for connecting near the auditory system of the tested object 5, activated upon receipt of an activation signal from the system of automatic activation of ABR channel, shown in FIG. 4.

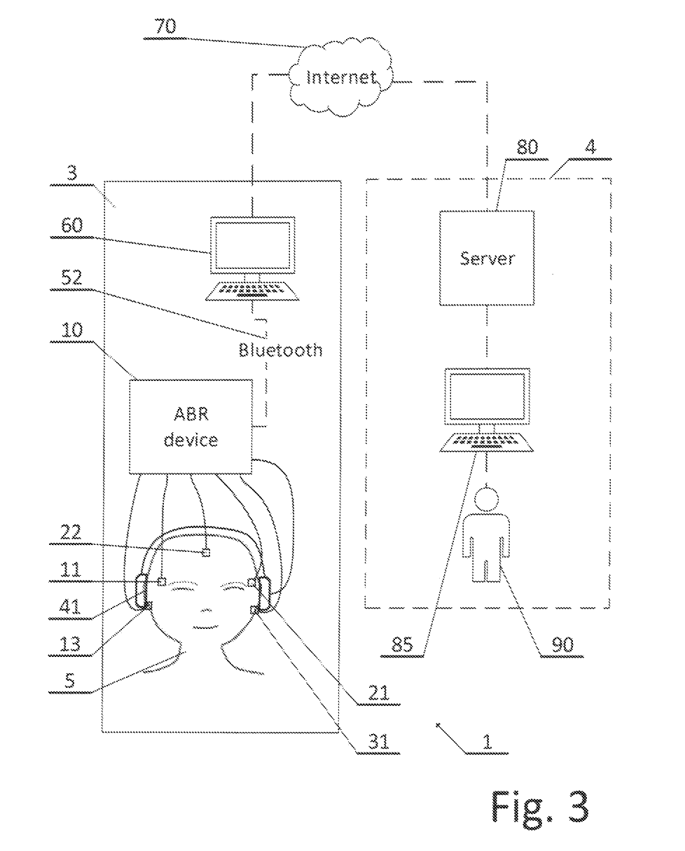

Furthermore, the ABR 10 device, which is a part of system 1, contains amplifiers for amplification of ABR signal up to 500 mV obtained from independent track of ABR signal collection induced by acoustic stimulation with audio track with acoustic device 41, for example headphones, and limiting frequency response using filters to a range of 0 Hz to 5000 Hz, ABR signal converter of independent channel for ABR signal acquisition from analog to digital, ABR signal recording system of independent channel for ABR signal acquisition in a configurable time interval measured after acoustic stimulation and a system for presenting status of ABR device and signals generated by ABR device 10 in graphical form, such as a display or screen 103, equipped with a control system, for example processor, shown in FIG. 4, to which commands are transferred via keyboard 104 or touch screen, and measurement results of ABR signal may be transmitted by the transmitting device 53 to unit 60 receiving and processing the data, shown in FIG. 3

If the processor of ABR device 10 has sufficient computing power, then ABR device 10 may be system 1, by which ABR signal with marked waves I-V after registration and processing is presented on the screen 103 of ABR device 10.

The system 1, shown in FIG. 3, allows the conduction of examination remotely at the optimum time of day or night, for example during physiological sleep, which contains the ABR device 10, operated by a control application installed on the ABR device and/or a unit 60 receiving and processing the data and intended for examined person or guardian at location 3 of tested object 5, for example on extra computer that communicates with ABR device 10 by external communication system, for example by using a Bluetooth 52 device shown in FIG. 4. In addition, the system 1 includes a central system 80 with a processing unit 85 and a screen for the person or persons operating 90 remote retrieval process and registration of signals for diagnostics of the auditory system and located in areas 4 outside of location of tested object 5, for example in a hospital or a testing facility.

The central system 80, in example shown in FIG. 3, communicates with the unit 60 receiving and processing data through the Internet 70 to which the results of signal measurements of ABR and EEG or EOA or EMG or their combination in the illustrated example are transmitted using Bluetooth 52 device.

The ABR device 10 allows the performance of the following steps, namely, impedance measurement of electrodes, acquisition of signal EEG or EOG or EMG or their combination, acquisition and ABR signal averaging, programming, calibration, and providing stimulus to person under examination, displaying basic information to user, such as the current mode or state of battery charge, two-way communication with the control computer via an interface, such as Bluetooth, using dedicated data transfer protocol.

A control unit of an example ABR device shown in detail in FIG. 4, is the processor 100, for example a microcontroller with 32-bit core ARM 7-AT91SAM7SE512 running at 96 MHz. The processor 100 is responsible for the control of all peripheral devices and communication services with the support computer 60, shown in FIG. 3, in case the processor does not have sufficient computing power. It comprises an efficiency of about 50 MIPS. Peripheral processor devices are: universal synchronous/asynchronous receiver/transmitter interface USART 54 used for communication containing a data transfer module, e.g. Bluetooth 52, serial bus SPI 34 which communicates with an analog-to-digital converter 33 of ABR channel 30 and programming audio codec, serial bus I2S 44 used for communicating with processor DSP and audio convener, and NAND FLASH 102 interface, used for recording current signal. The most basic version of a processor contains 32 kB of primary storage (types of RAM) 101 and 521 kB internal program memory. For recording real-time ABR signals, NAND FLASH NAND512W3A 102 with 64 MB memory has been used, which is enough to store about 20000 responses of ABR, or EEG or EOG or EMG signals or their combination.

The processor provides the ability to perform ABR signal recording remotely and other actions such as device monitoring or software updates using the screen 103 and keyboard 104. In this system, it is possible to implement support to all features that are necessary for the remote control device. In addition, to be remotely managed, it is equipped with appropriate communications interface circuits 50 of external communication with the transmitting device 53, which transmits data to the unit 60, shown in FIG. 3, which receives and processes data. As a basis for this interface, a removable communication module is used. Design of the device allows for the use of two types of wireless transmission, such as Bluetooth 52 and Wi-Fi 51 or other 153. In the illustrated example of execution, by default communication is carried out via Bluetooth, and the device's design allows plugging in a Wi-Fi module instead of a Bluetooth module to enable device connection with a larger distance between devices, e.g. in case of separate floor apartment. Communication via Bluetooth allows signal transmission up to 10 m. In the case of communication using a Bluetooth module. Bluegiga WT12-A-AI unit has been used, which operates full communication and communicates with the processor via USART. The computer connects to the device via the line 54 using COM port interface. Data transmission is carried out at a rate of 230400 baud. In the case of communication with computer via Wi-Fi 51, transmission is carried out via a Wiznet WizFi 210 module. This module contains embed, complete TCP stack and the communication is carried out with use of a standard router, which has Wi-Fi support. An example of transmission speed is 230400 baud, and communication with the computer proceeds via COM port interface, thereby modules can operate interchangeably and completely transparent to the controlling application. Allowing the selection of the version of the device based on specific needs is an advantage and flexibility provision of the device. Using this system of components, namely to operate the device communication with a computer controlling it by Bluetooth and providing communication of the control application with a central server of the central system 80 shown in FIG. 3, provides the opportunity to carry out both remote operations on the device and the update of the ABR device software 10.

One of three channels of ABR device 10 is an independent ABR signal channel, in short ABR channel 30. Initial components are amplifiers and filters, including instrumental operational amplifier INA333, which provide a high level of common signal attenuation. Differential amplification of stage is 34 dB, which provides signal processing in the presence of a differential constant signal up to 100 mV, which corresponds to the skin-electrode potential from electrodes 13, 22, 31. The next stages of amplifiers and filters 32 provide further amplification of the signal and reduce bandwidth, which are formed at AD8609 system with a second-order filter. Bandwidth of the amplifier is set 0 Hz-5000 Hz. Amplification of all ABR channel is 51 dB, which in combination with 24-bit converter gives a resolution of 0.6 nV/bit. Signal conversion from analog to digital is carried out with analog-to-digital convener 33 in the above-mentioned 24-bit converter ADUA1761. It is a specialized audio processor that can convert a signal and calculate digital acoustic signal filters in real-time. Within ABR device 10 it is possible to single out the system to measure the ABR signal induced by acoustic stimulation. The system to measure the ABR signal induced by acoustic stimulation includes automatic activation system or a circuit 106 for automatic activation 106 of the ABR channel with an input 39 of activating signal, which communicates by the output 29 of a system 105 informing about sleep phase occurrence to inform that sleep phase has been observed and previously mentioned independent channel 30 for ABR signal acquisition with electrodes 13, 22, 31 used for connecting in the vicinity of the auditory system of tested person, activated by input 35 of line 36 after receiving activating signal from output 37 of the automatic activation of the ABR channel circuit 106.

To acquire the signal used for the detection of appropriate sleep phase to perform an ABR test, an independent signal channel EEG or EOG or EMG or their combination is used, which is built on a specialized ADS1298 system, in short EEG or EOG or EMG channels or their combination 20 with the output 25 forming part of the system for measuring EEG or EOG or EMG signals or their combination with the system 105 connected by a connection line 26 to the output 25 and informing that sleep phase has been observed. The system 105 informing about sleep phase occurrence has the input 27 and the output 29 to communicate with the independent signal channel EEG or EOG or EMG or their combination and the independent measurement channel 20 of EEG or EOG or EMG signals or their combination is a complete signal channel with analog-to-digital converter 23, such as 24-bit. The processor communicates with a system via independent EEG or EOG or EMG channel or their combination using the SPI 24 bus. In addition, the converter ADS1298 performs measurement of electrode contact resistance with skin and allows for RLD signal production, which increase signal attenuation appearing as line interference.

The PCM3010 system is also responsible for generating of the acoustic stimulation signal with the use of acoustic stimuli channel (audio channel 40). Signal from the output of the converter signal generator 43 is amplified to an appropriate level by a signal amplifier, such as specialized power amplifier TPA6110A2. Audio system is double ducts, which allows simultaneous conduction of measurements for both channels. Stimulation signals transmitted to the audio transmission system 41, such as headphones, are generated by the control processor via the I2S bus 44. Acoustic signal stimuli channel supports formation of any sound, capable of being recorded in 24-bit signal, such as tone of 1000 Hz or 500 Hz crash. To achieve the required level of acoustic signal stimuli, an additional amplifier 42 is applied, realized in the application system TPA6120A2.

In order to ensure an adequate voltage level for each system device, power supply 55 and 38 with power lines have been applied. Power supply contains independent stabilizers for analog amplifiers, digital circuits and analog output amplifiers. This design ensures noise reduction in the measurement signal from the cooperating device parts. In the presented invention, the device is powered by rechargeable batteries or accumulators which can be charge in an external charger, for example, two AA batteries with no wired connection to other devices.

It affects reduction of the occurring interference network signals and simplifies design for safety reasons. Power supply can be realized by step-up voltage converters, for example MCP1640 systems, increasing the voltage to required levels, which are 3.3 V and 5V. Audio amplifier channel contains an independent part of the power supply to protect from signal interference caused by power lines. Analog input amplifiers are powered by 5V. Higher power range is required in order to ensure the high gain of the first degree which increases the attenuation of common mode. Power supply of the analog part of the system is performed by MAX1595 system-capacitance converter in the application system.

The ABR device can be placed in a casing, which can be a casing available on the market or in a dedicated casing. For connecting the electrodes 11, 13, 21, 22, 31, which are attached at specific sites of the tested object, a 1.5 mm touch-prof, slot has been used. These sockets are widely used in connections of EEG electrodes, which provides versatility and the ability to connect to commercially available electrode cables. In the configuration shown in FIGS. 2, 3 and 4 the ABR device consists of inputs for five electrodes, namely the electrode L 31 located on the left mastoid process, the electrode R 13 located on the right mastoid process, the electrode L EEG 21 located on the left temple, R EEG electrode 11 fixed to the right temple and electrode N 22 placed on the forehead.

The standard mini-jack audio connector has been used as an audio output for acoustic stimulus emission in the example shown in FIGS. 2, 3 and 4. It is compatible with most headphone models including the most popular audiometric headphones such as Sennhaiser HDA200. In one case the ABR device has been equipped with a screen 103, for example, in monochrome, binary, DOGS102 graphical LCD display with a resolution of 102×64 pixels located on the front of the package. Owing to the help of the screen, the device is able to inform the user about the current operating status and about the basic values of parameters. After connecting another screen it is possible to show a graphical representation of ABR or EEG or EMG or EEA, or their combinations.

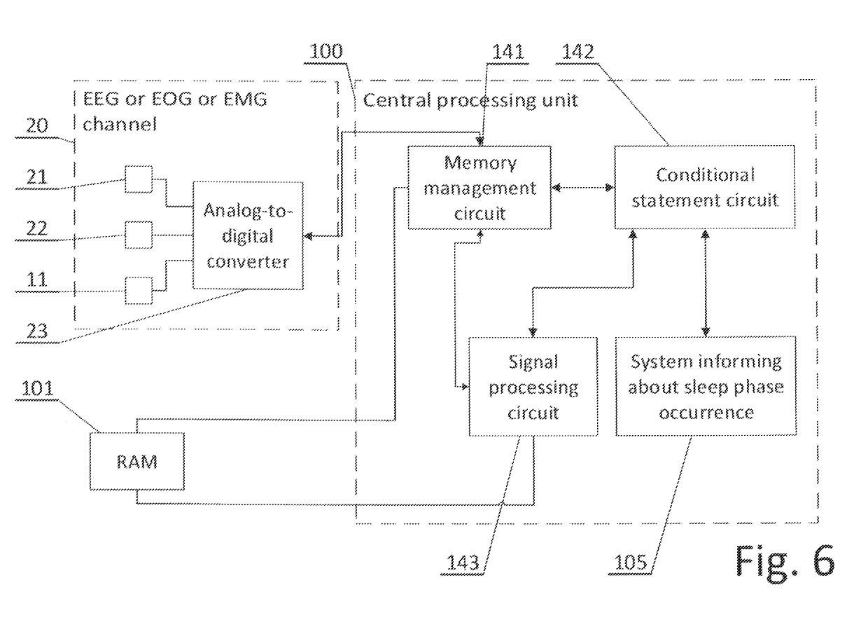

FIGS. 6A, 6B and 6C show a block diagram illustrating the processes of ABR device communication with control application accounting for software which is an intermediary between an ABR device and the central system. Communication processes are designed to provide the sequence of events in the system, but they have no representation in real names of commands at the level of communication protocol. In the example shown in FIG. 2, this software has beers installed on the user's computer. This application can perform automatic and manual models of measurement and implement full service facilities. In order to ensure transparency and simplicity in the user's interface as well as maximum automation, the default operating system is a Microsoft Windows 7 and other recent versions, but this does not exclude the use of other systems.

After registration of signals used for the diagnostic of an auditory system with the ABR device for predetermined recording parameters, which can be type of stimulus, polarity, number of averages, the repeat rate and data receiving channels, the system processes the signals and treats them because in addition to the ABR signal, waveforms include the interfering signals because the electrodes collect not only the ABR signal, but also all the potentials occurring in the body.

FIG. 5 shows schematically a noise amplitude spectrum 201, 202, 203, 204, 205, 207, 208 and also ABR signal spectrum 206. It is clearly shown that the ABR signal is located almost entirely in the noise and there is no possibility to recover the ABR signal by simple band-pass filters. The most important source of artifacts is the brain (EEG) generating interference 203, 204, eyes (EOA) and (ENG) that produce interference 202, the heart (ECG) and skeletal muscles, especially the neck and head muscles (EMG) that produce interference 207, which are described in more detail below. Electroencephalogram shows the electrical activity of the brain. In the case of a sleeping person the amplitude is about 400 uV and the dominant frequency is about 10 Hz. During standby mode amplitude value is 70-100 uV, and the frequency band is 3-40 Hz. The amplitude of interferences generated by the eyeballs movement and the optic nerve is 400-1000 μV and a frequency range is 0.5-10 Hz. Artifacts made by eyeballs movements have a very large amplitude in comparison to ABR signal, and may cause saturation of the amplifier. The amplitude of interferences generated by the heart (ECG) measured at the scalp is about 500 μV a frequency range is 1-50 Hz. Due to the smaller distance between head and heart, the amplitude may be up to 800 microvolts in children. Frequency band is also wider. Another source of artifacts is muscle activity which generates very strong interferences, whose amplitude varies between 100-500 μV. Muscle artifacts always appears in people during standby mode. These artifacts mostly disappear in a physiological or induced sleep, hence the ABR measurement was usually carried out during sleep. It turns out that muscle artifacts may occur even when there is no body movement. Frequency range of EMG is about 30-500 Hz. which largely overlaps with the frequency range of the ABR signal from 30 to 3000 Hz. Overlapping of the two frequency ranges means that muscle artifacts are the most damaging and difficult interferences to filter out. Appearance of muscle artifact at the end of the measurement may lead to a complete distortion of ABR signal and the necessity to repeat the measurement. To remove the effect of muscle artifacts we can use special techniques of numerical signal processing and repeatedly, for example from 500 to 2000 times, repeat the same measurement as shown below in figures presenting flowcharts of appropriate systems and procedures for the registration and signal processing relating to the diagnostics of an auditory system. Furthermore, there is a lot of interference on input of the differential amplifier 208 caused by electric and magnetic fields from devices located nearby the amplifier as well as radio frequency fields, which are also shown in the chart. Interference from power lines have a frequency of 50-60 Hz and their multiples. Power lines induce currents in the transmission cable and in the device itself. Electric and magnetic fields which occur in the clinical environment usually have a much higher intensity than the average intensity of the earth's field. This is a result of the presence of a large number of medical devices that are usually not shielded, for example MRI, and the presence of power supply devices. Another disadvantageous factor is that the radio-frequency interferences can interfere with each other, resulting in a low frequency lying in the band 30-3000 Hz, which coincides with the ABR signal band. FIG. 5 shows that the most damaging interferences are muscle artifacts and in particular 203, 204, made by the brain (EEG) and the interference 202 derived from eyeball movement. Filtering network and interferences induced by radio frequency waves is somewhat simpler to implement, and systems used for this purpose are known.

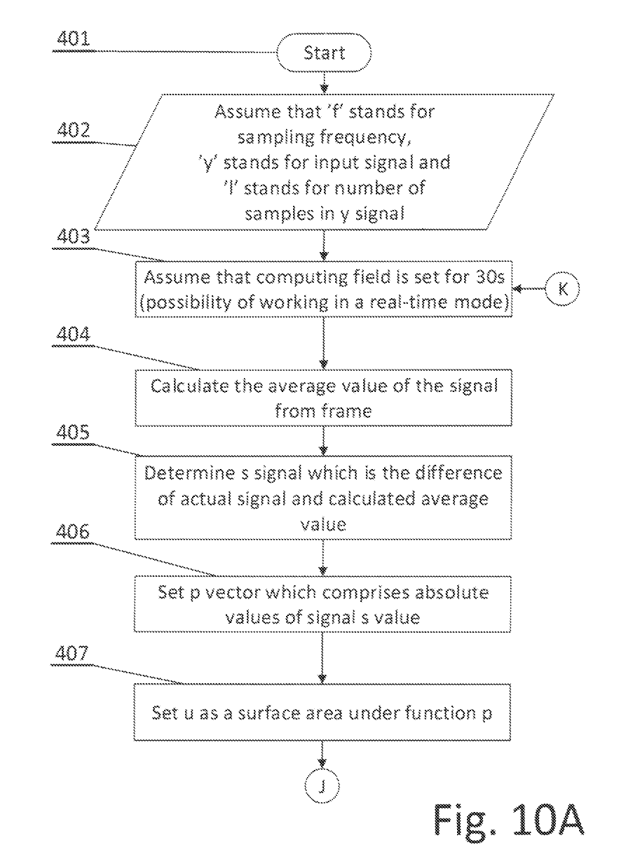

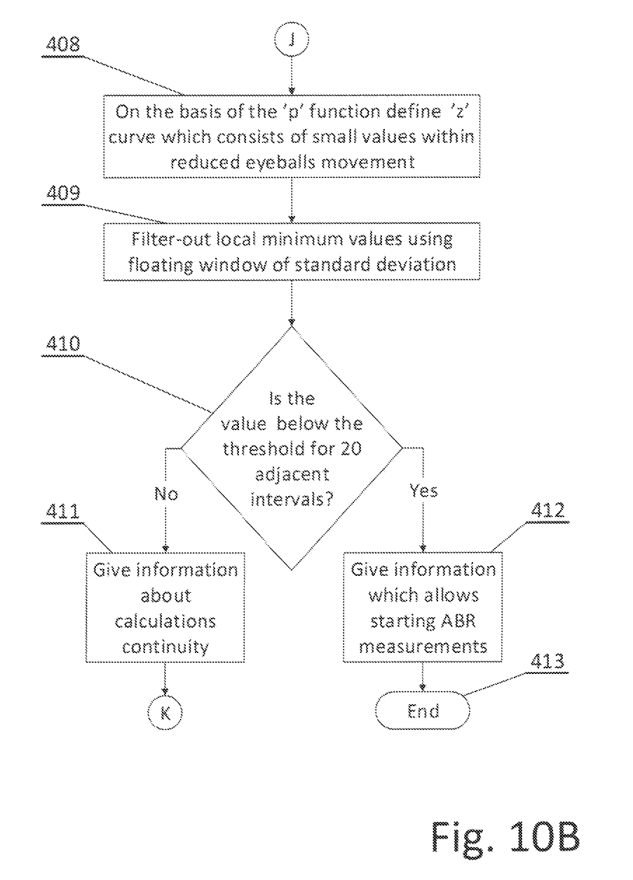

FIG. 6 shows the system for measuring of bioelectrical brain activity signals or eyeball movement or muscle twitching or their combination (EEG or EOG or EMG) and the detecting threshold below which a decision regarding the start of ABR testing is made automatically according to the procedure described below in relation with FIGS. 10A and 10B. The system contains an independent EEA or EEG or EMG circuit 20, or their combination with the electrodes 11, 21 and 22 and the analog-to-digital converter 23, with the RAM memory 101 and dynamic memory allocation system 141, communicating with the EEA or EEG or EMG channel, or their combinations 20, RAM memory or RAM 101 and a signal processing system or signal processing circuit 143, which communicates with RAM 101 and conditional statement circuit 142, which communicates with the system informing about sleep phase occurrence 105. System informing about sleep phase occurrence 105, dynamic memory allocation system or memory management circuit 141, conditional statement circuit 142 and signal processing circuit 143 may be implemented on separate processors or on central processing unit 100.

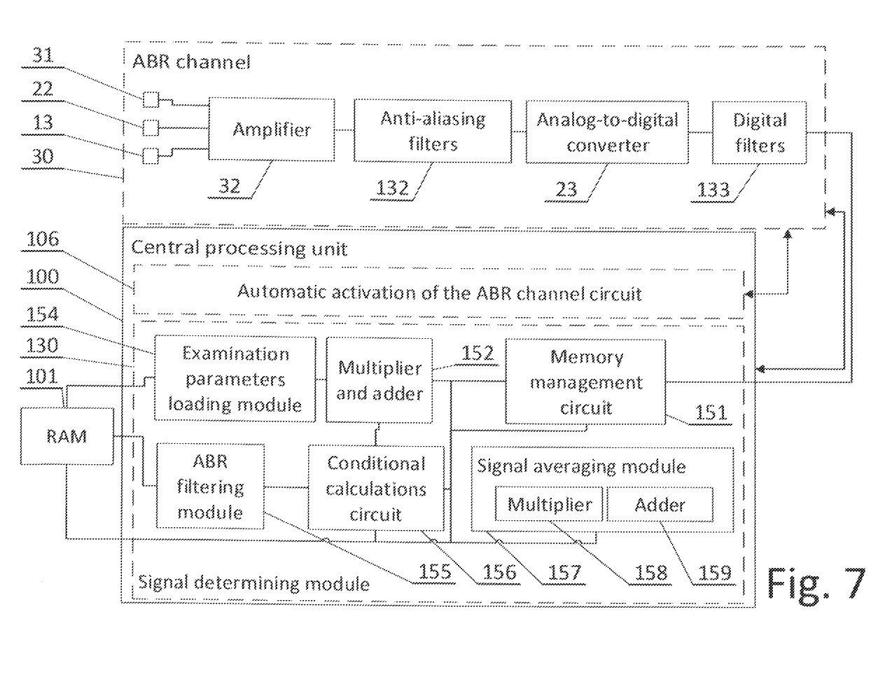

FIG. 7 shows automatic activation of the ABR channel circuit containing ABR channel 30 with electrodes 13, 22, 31, 32, amplifier 32, anti-aliasing filters 132, analog-to-digital converter 23 and digital filters 133. ABR signal acquisition system also includes RAM memory or RAM 101, automatic activation of the ABR channel circuit 106 and ABR signal determining module or signal determining module 130. ABR signal determining module or signal determining module 130, shown in FIG. 7, contains memory management circuit 151, the multiplication module or multiplier and summation module or adder 152, examination parameters loading module 154, ABR filtering module 155, the conditional system or conditionals calculating circuit 156 and signal averaging module 157 with multiplication module or multiplier 158 and summation module or adder 150. Automatic activation of the ABR channel circuit 106 and signal determining module 130 can be implemented on separate processors or processor 100.

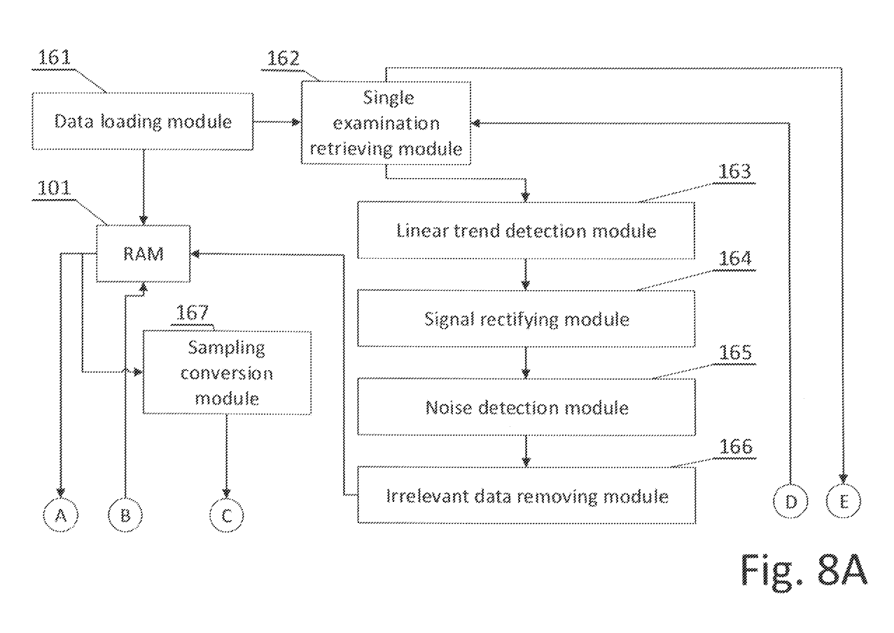

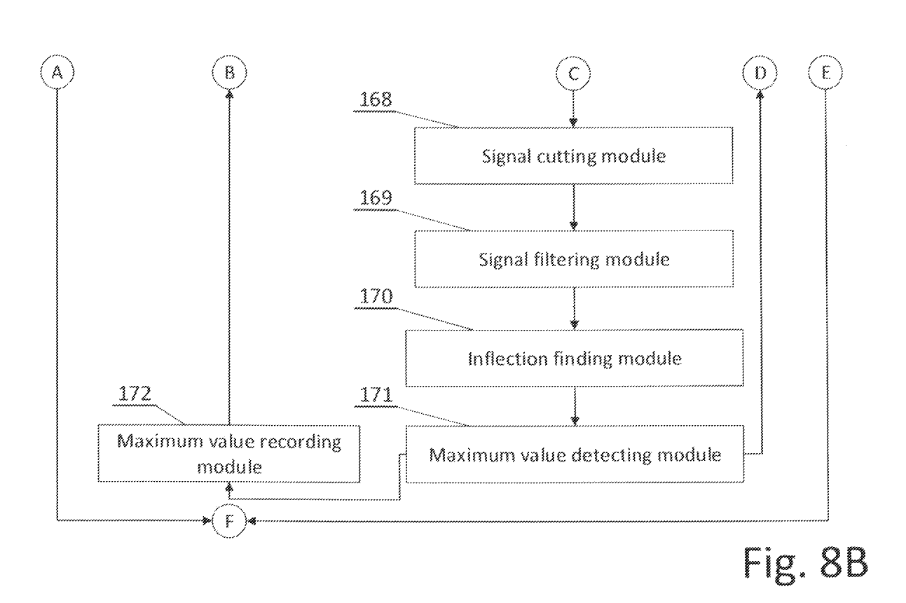

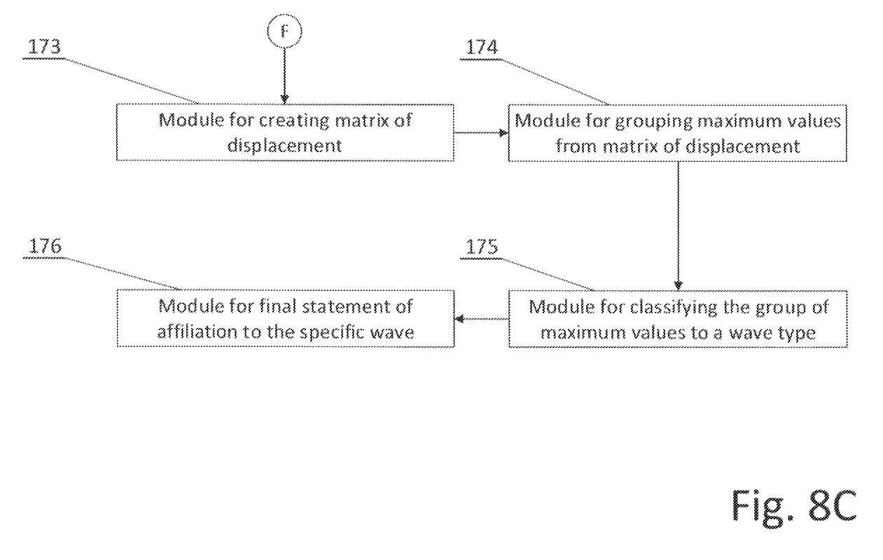

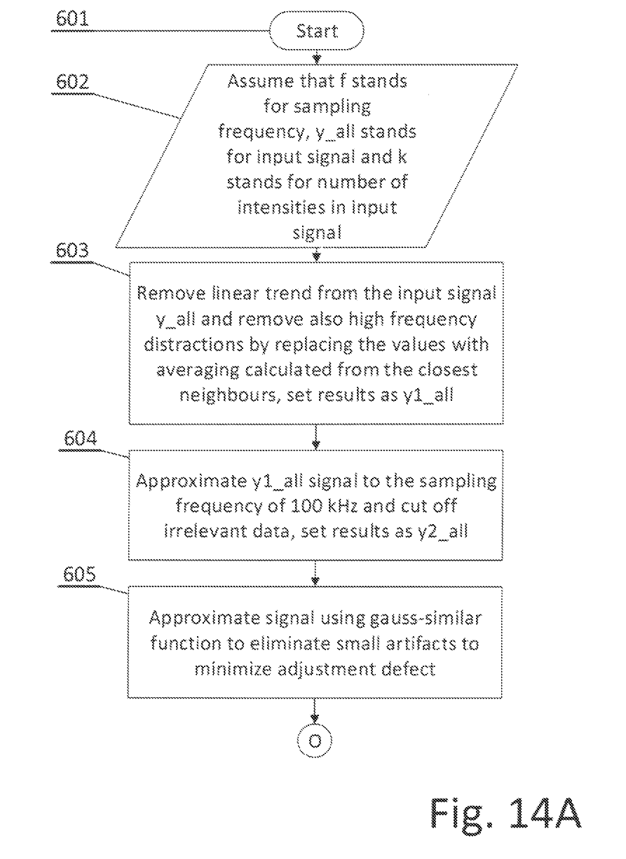

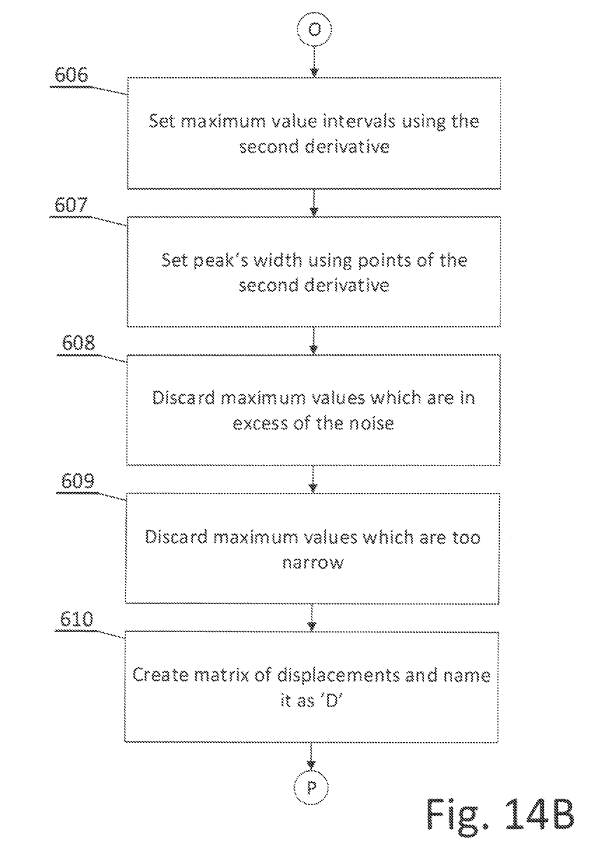

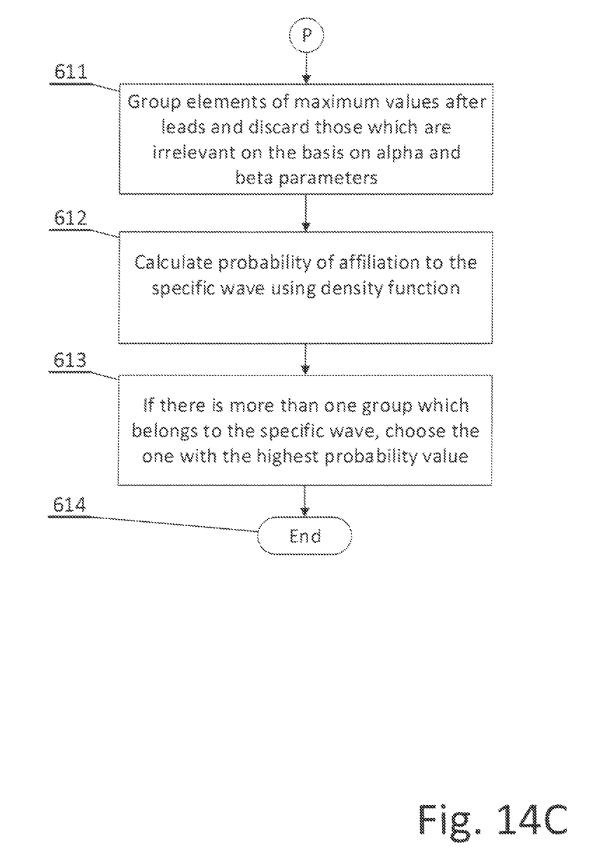

FIGS. 8A, 8B and 8C show the system for detecting waves which occur in the ABR signal, according to the procedure described below, in connection with FIGS. 14A, 14B and 14C, which includes modules described below, which may be implemented in separate processors or processor or central processing unit 100 shown in FIG. 4. One of them is data loading module 161, which communicates with the RAM memory or RAM 101 as shown in FIG. 4 and with a single examination retrieving module. The main role of data loading module 161 is to load signal sampling frequency, the input signal consisting all of the measurements taken for various intensities and information, such as the number of intensities in the input signal. Single examination retrieving module 162 communicates with a linear trend detection module 163 or a single examination run of ABR signal in order to fit it into a straight line expressed by the equation y=ax+b, where ‘y’ represents the value of the trend function, ‘x’ represents the next time value measurement and ‘a’ and ‘b’ are parameters for the measurement. Module 163 communicates with signal rectifying module 164, by which from a single signal the linear trend is subtracted. The result of this operation is to rectify the signal. For example, if the signal increases with time, this effect is removed as a result of the operation performed by the signal rectifying module 164. Another system module used for the detection of occurring waves is noise detection module 165, which calculates noise to signal ratio, and irrelevant data removing module 166, that in the course of a single measurement verifies each amplitude value ensuring that the value is not four times higher than noise to signal ratio—the noise to signal ratio value calculated by the noise detection module 165. If the amplitude value is four times higher than the noise to signal ratio, then it is converted to the average from the two nearest values, which are below the threshold. In this way, the irrelevant data removing module 166 removes single large peaks which represent noise. ABR signal after noise removal is recorded in RAM memory or RAM 101, which communicates with the sampling conversion module 167, by which the signal ABR is resampled on the sampling frequency of 100 kHz, by an approximation of the neighboring points with the straight line connecting two points. Sampling conversion module 167 communicates with signal cutting module 168, reduces each signal longer than 10 ms in value. This means that the redundant samples, such as those which have more than 10 ms are rejected from the signal by the signal cutting module 168, which communicates with a filter signal module 169, which approximates the signal using a gauss-like function that eliminates small artifacts so the matching error is as small as possible. Signal filtering module 169 communicates with inflection finding module 170, which calculates inflection points by using a second derivative, and also communicates with maximum value detecting module 171. Using inflection points module 171 can estimate width and height of the peaks in relation to these inflection points. To determine the specific point as a maximum value, its width must be appropriate and its height must extend over the range far enough beyond the multiplicity of noise approximation. Peak width is calculated by the distance between inflection points of trailing edge and rising edge. The maximum value detecting module 171 removes maxima which insufficiently exceed noise and those that are too narrow. The correct peaks are recorded in RAM 101 by maximum value recording module 172, which communicates with maximum value detecting module 171. Module for creating matrix of displacement 173 communicates with RAM memory 101. Module 173 creates a square matrix whose values are the differences between the maximum peaks assigned for different values. The statistical method shows that difference in peak position between actual maximum and the maximum at lower intensity should be in the range from −0.13 to 0.93. This method allows to create a transfer matrix where elements which satisfy criterion have time values and the other elements are take on infinite values. Module for grouping maximum values from matrix of displacement 174, whereby the maxima of the group are created by selecting the minimum values of the transfer matrix, communicates with the module for creating matrix of displacement 173. Considering all maxima for ABR measurements at different intensifies, the module 174 recognizes and groups only maxima, which differ between each ether in a relatively small time interval. The module 174 communicates with the module 175 for classifying the group of maximum values to a wave type, which calculates the grouping of the wave using a probability density function. The algorithm calculates the probability of belonging to the waves I, III, V or 0. The highest value of probability means getting the label of the group. Group classified as group 0 is removed. The module 175 communicates with module for final statement of affiliation to the specific wave 176. When the label, that is wave I, III, V or no wave is allocated only once, the result is returned in the same form. If it happens that two or more groups get the same label, the one with a higher probability of belonging to a particular label is allocated to that label. Other groups connected under this label are removed. Thanks to described modules, a set of plots 620 is created, and it is shown in FIG. 15, for example, decreasing acoustic signal intensity. The above-described modules are electronic circuits, such as adder, differentiator and integrating circuits, which are well known in the art.

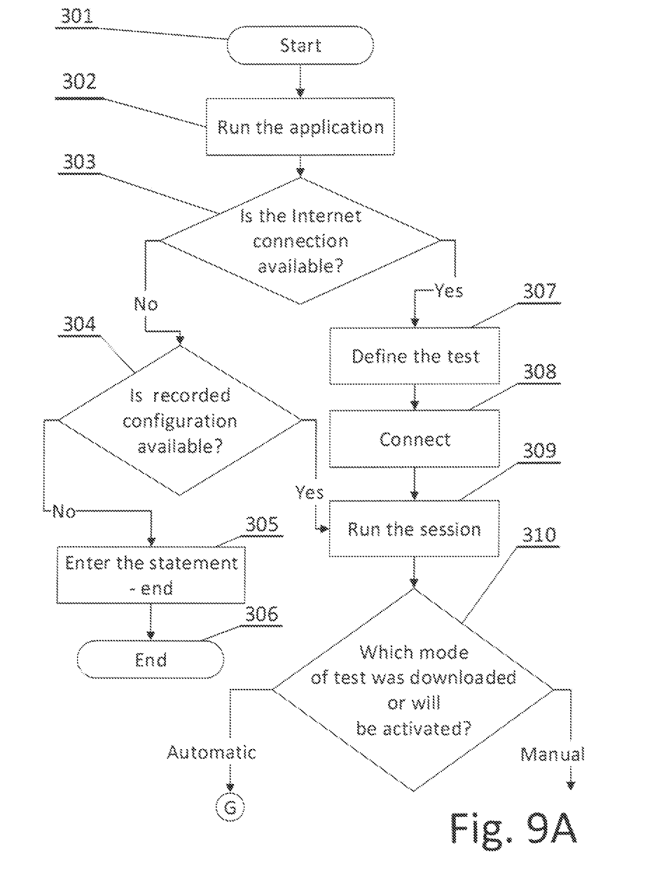

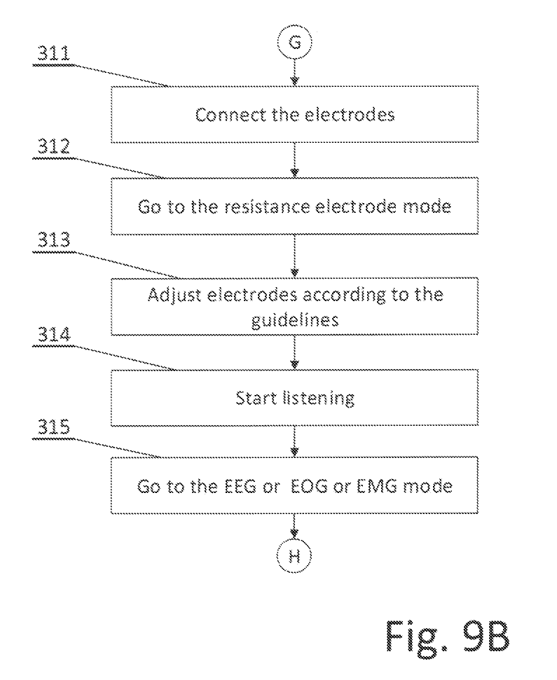

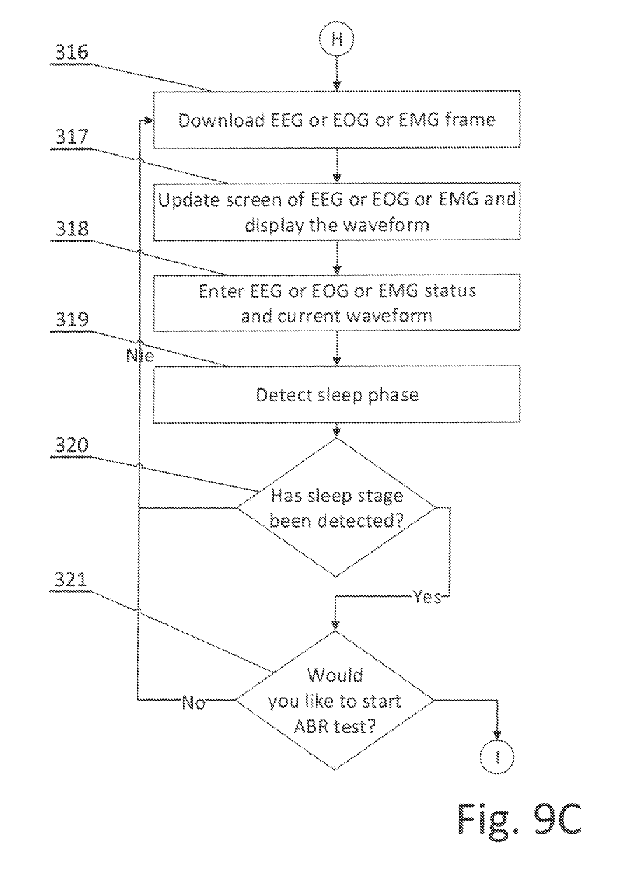

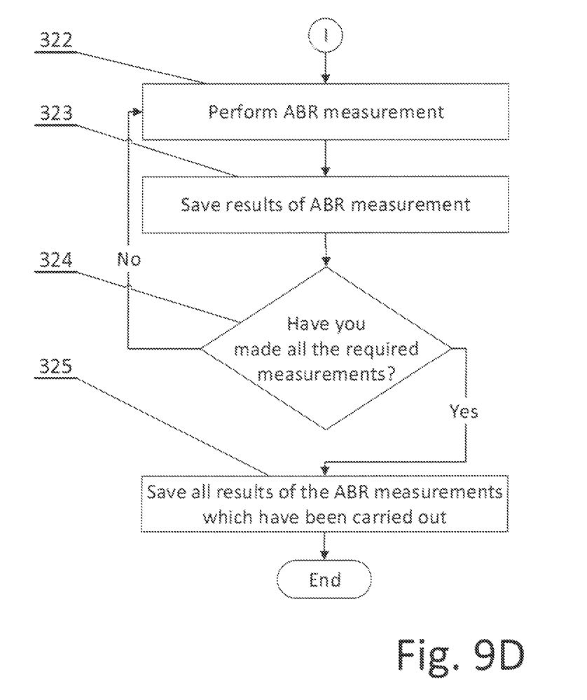

According to FIG. 9A after beginning in step start 301, the application starts in run the application 302. In step 303 application checks if there is an internet connection available, in the case where the user has no access to the internet, in step 304 it is checked whether saved configuration of measurement or information pertaining to the person to be tested is available. When the configuration has been saved, the measurement session is started, otherwise in step 305 information about ending the application is issued. Then in step 306 the application is ended. However, if the user has access to the Internet, he gives an address which he received from administrator with ABR device and the license for carrying out the ABR measurement. The user also gets an access code generated only once by the central system, using this code allows to link the device ID and the person to be tested and conduct an application's authentication of the device in the central system. In step 307 the user inputs address and access code on initial configuration screen for defining the examination. When the user clicks the navigating button to the next screen, he can see information about the ongoing configuration of the application with the central system and that necessary actions are performed at the level of communication and configuration of the application with central system after obtaining connection in step 308. The result is downloading of that application configuration to the current examination session from the central system and the data of the tested object assigned to the device number in the central systems. From this stage, an application status oar displays information for the tested person about device connection status, current configuration and availability of the central system. After configuration of the application with the central system, download of tested object data and registration of session, the startup screen appears and the session stabs in step 308. The startup screen displays easy instructions needed to properly connect electrodes and to launch the device in order to perform registration. Status of device connection is being updated on a regular basis so that the user can easily tell if he has completed all steps that are necessary to start the registration. In step 310, the mode of ABR signal recording is determined. If automatic registration was chosen, the application displays screens appropriate for user working without assistance of technician performing registration. Screen one displays preparations to start. On this screen, the user is briefly instructed in steps 311, 312, 313, as shown in FIG. 9B, on how to properly fasten electrodes to impose the least possible resistance. In addition, indicators of electrode connection quality are displayed for the user. When the user properly connects the electrodes and clicks the button navigating to the next screen, the user will see information that the device is working in automatic mode and additional actions are not needed. A switch to listening mode takes place in step 314. The default mode that device enters into in step 315 after connecting electrodes is EEG or EOG or EMG mode or their combination, at the beginning of which in step 316 is displayed EEG or EOG or EMG frame, or their combination, and takes place an update of EEG or EOG or EMG screen, or their combination, and displayed is graph in step 317, shown in FIG. 9C. After that, in step 318 is checked the status of EEG or EOG or EMG or their combination, and is displayed on the current graph. During EEG or EOG or EMG mode or their combination, in step 319, is detected optimal sleep phase to start examination and graph EEG or EOG or EMG or their combination is updated on a regular basis. The application continues its work even when the main window is closed. After sleep phase detection in step 320, application switches to the next screen which informs about which current automatic step of the test is being performed. A decision about starting the ABR test is made automatically in step 321 and its beginning is initiated in step 322 shown in FIG. 9D. During the test, the user has to select the recording parameters, which can be type of stimulus, polarity, number of averages, frequency of stimulus repetition, and channels. Selected recording parameters are listed below:
Type of stimulus;
   Click
   Tone of 0.5 kHz
   Tone of 1 kHz
   Tone of 2 kHz
   Tone of 4 kHz
Polarization
   Positive
   Negative
   Alternate
Number of averages
Stimulus repetition, which means number of stimulus repetition per second
Channel(s)
   L
   R
   L+R

In the example implementation shown in FIGS. 9A, 9B, 9C and 9D during the acquisition of the ABR signal conducted from the step 322, there is information displayed on the screen to the user, namely the current channel on which the acquisition is performed, the current intensity of the stimulus, the current ABR waveform is being refreshed from ABR device in a real-time.

Similarly to the previous screen, it is not necessary to have the main window active during the acquisition. In step 322 the single measurement of the ABR is performed, and the result of this measurement is saved in step 323. After completing all the required measurements of the ABR, as stated in step 324, the results of all performed measurements of ABR are saved in the step 325, after which the ABR examination finishes. After clicking the button ending the ABR examination, data is sent to the processor or/and the supplementary computer or/and the central system, and the user can exit the application.

During the operation of ABR in manual mode, in contrast to the automatic mode, the technician or the person who operates has insight in the device working in real-time and gives all the commands manually on the server. Commands are received with the use of a communication protocol by the operating application, then they are translated and transferred to the ABR device. The expectant screen displays the proper information about the current state. While establishing a connection with the technician, the user is automatically redirected to a screen about preparing for operation. This screen is similar to the automatic mode screen about electrode resistance. The difference here is that if necessary the technician can instruct the user on how to place electrodes and how to improve quality of their attachment. When the technician ascertains that the electrodes are attached sufficiently well, the user is redirected to the screen with preparation to examination, and then follows the acquisition mode of signals: EEG, or EOG, or EMG or their combinations. From now on, the user, similarly like in automatic mode, can close the main window of the application. Both technician commands, and acquisition and transmission of data from the device are performed in the background. When the technician sets measurement parameters and redirects the device into the ABR acquisition mode, the application redirects the user to the next screen. Just like in an automatic mode, the semen displays basic parameters of measurement which is currently going on, such as channel, intensity, as well as other similar parameters. The technician can switch the device between modes: EEG, EOG, EMG, their combinations and ABR at any time, which makes it possible to navigate automatically between screens many times. Just like in automatic mode, after completion of the examination, the user is redirected to the summary screen and after clicking the button terminating the examination, data is transmitted into the central system and the user can exit the application. The quality of acquisitioning signals for diagnosis of the auditory system using the ABR device is influenced by noise caused by occurrence of muscle artifacts, which during the sleep are very small or all together disappear. Research has shown that the best phase of sleep to perform the acquisition of auditory brain stem response is a deep sleep, the 4^{th} phase. It was stated, however, that the execution or signal recordings for diagnosis of the auditory system is also possible in the 2^{nd} and 3^{rd} phase of sleep. In FIGS. 10A and 10B in the form of an algorithm using a block diagram, a method for detecting the phase of sleep is shown, which is carried out before the registration of signals for the diagnosis of the auditory system. For sleep detection, it was assumed that the ABR device should detect the 2^{nd}, the 3^{rd} or the 4^{th} phase and be easy to operate, which results in a short period of sleep phase detection, cope with a limited set of information, use the minimum number of electrodes, as well as make it possible to analyse the signal in a real-time and return the information whether to start automatically the acquisition of signals for the diagnosis of auditory system using the ABR device or not. On the basis of studies, it is accepted that a sufficient signal for sleep phase detection are measurement of the following signals: horizontal EEG, horizontal EOG, which are induced by eyeballs movement or EMG signal or their combination.

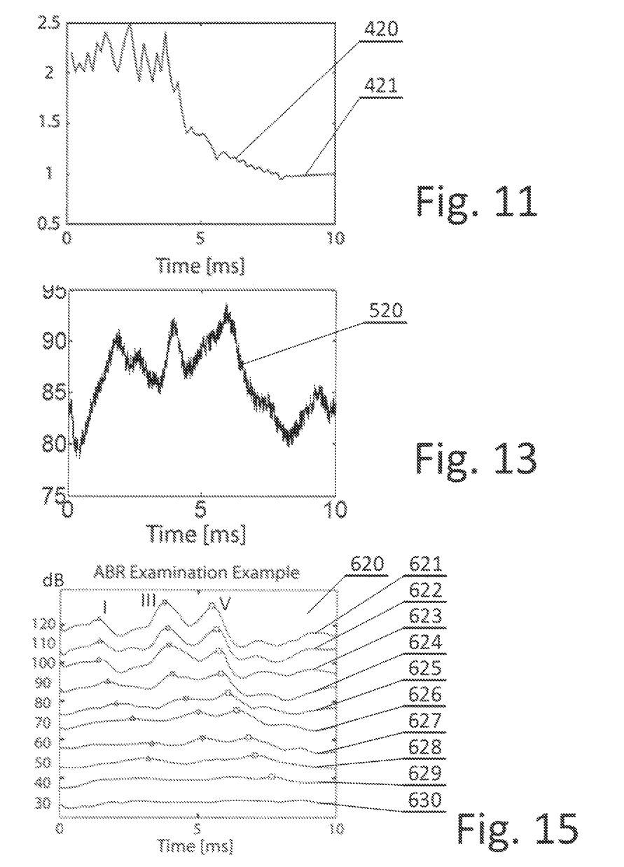

According to FIGS. 10A and 10B sleep phase detection begins from the start at step 401. In the step 402, the value of sampling frequency f, e.g. 100 Hz, and the number of samples are determined and then the acquisition of the input signal y from electrodes begins. In the step 403, the value of the computing window, which is the time interval, is determined, e.g. 30 seconds. After the lapse of the first interval, the average value of the signal obtained from electrodes which measure the horizontal movement of eyeballs is calculated in the step 404. In the step 405, the signal s, which is the difference of the actual signal and the calculated average value, is determined. Next, in step 406 the vector p, which consists of absolute values of signal s is determined. Step 407 is calculating the area under the function p and setting it as u, and then in the step 408 on the basis of function p, the curve comprising small values within reduced eyeballs movement is determined, knowing that the resulting curve contains very low values in the case of the occurrence the 2^{nd}, the 3^{rd} or the 4^{th} phase of sleep, which indicates the area in which the ABR examination can be started. According to the fact that during the day the activity of eyeballs is sometimes low and the waveform of EOB after above transformations also has small values in a form of single downward peaks. In step 409 local minimum values are filtered out by a moving window of the standard deviation. In step 410 it is checked whether a predetermined number of intervals from the past, e.g. 20 of them, is below a specified threshold. The threshold is chosen empirically after conducting experiments. It turned out that the received part 421 of the waveform 420 shown in FIG. 11, is often below the value of 5000 in the time of the occurrence the 2^{nd}, the 3^{rd} or the 4^{th} sleep phase. After detection of the sleep phase, in step 412 information about its detection should be given. The detection of sleep phase terminates during step 413, whereas the sleep phase can also be another, optimal moment for starting an ABR examination. Due to the fact that the information about the number of windows from the past is needed, the earliest possible time counted from plugging in the device to starting the registration of signals for diagnosis of the auditory system with the usage of the ABR device is 14 minutes and 30 seconds. Detecting sleep phases itself is based on the methods known from the state of art, for example from the publication: ‘Human Sleep and Sleep EEG’ written by K. \check{S}u\check{s}máková, and the best time to start an ABR examination is the moment of obtaining the value below the previously described threshold. If the phase of sleep is not detected, in step 411 information should be given the about proceeding with the examination and the application should go to step 403 in which the new computational window is determined.

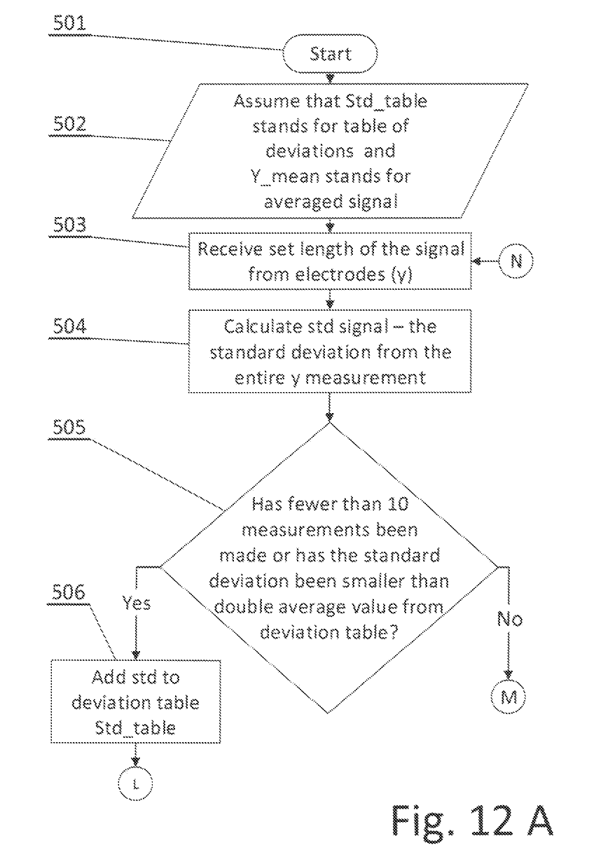

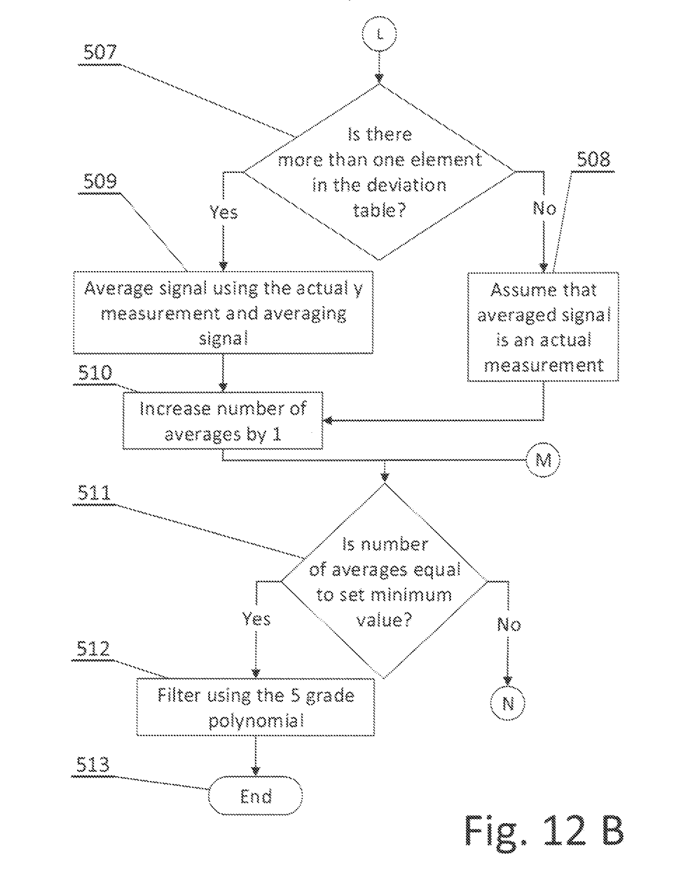

The registration of signals for diagnosis of the auditory system itself using the ABR device, regardless from the mode in which it works, is carried out as shown in the flowchart in FIGS. 12A and 12B. After starting in step 501 and defining the size of the table of standard deviation Std_tabel, the mean value of signal Y_mean is defined in step 502. In step 503, measurements of signal y are drawn from the electrode or electrodes and is executed in a strictly defined timeframe. In step 504 standard deviation std from the whole measurement of the signal y is calculated and in step 505 is checked if less than ten measurements were performed or if the standard deviation is less than double the average from the table of deviations. In the case when less than ten measurements were made or if the standard deviation is less than double the average from the deviations table, in step 506 deviation std from the current registration is added to the table of deviations as one of the elements of the Std_tabel table. In step 507 it is cheeked if in the table of deviations Std_tabel there are saved more than one element of the table and in case when there is saved only one element, then in step 508, it is assumed that the average signal is equal to the current registration of the signal. Otherwise, in step 509, average signal is calculated with the use of current registration or the measurements of signal y and average signal, while increasing the number of averages by 1 in step 510. On the other hand, in step 511 it is checked whether the number of averages is equal to the predetermined minimum value and in the case when the number of averages is equal to the predetermined minimum value, then in step 512 the signal is filtered using the polynomial of the fifth degree and in the step 513 the registration of signals for diagnosis of the auditory system using the ABR device for determined registration conditions is terminated. This kind of data processing compresses a signal more than 31-fold, and the filtered signal is resistant to single, very large, muscular artifacts. This is due to the application of a polynomial of the fifth degree that does not include minor changes which include large amplitude. So the solution immunizes the final result from existing noise, including noise generated due to high background noise, and exposes the right signal, which can be located in the noise very well. When the number of averages is less than the predetermined minimum, in step 503, a next measurement of signal y derived from the electrode or electrodes at a predetermined time is performed, and the cycle of registration is repeated.

As it has appeared from the measurements, the possibility of separating the ABR signal 520, shown in FIG. 13, comes from the fact that it is correlated with the stimulation and very poorly correlated with the disturbances. The same procedure of ABR signal separation is shown in FIGS. 14A, 14B and 14C. After starting in step 601 and defining sampling frequency f, the input signal y_all and the number of intensities k contained in input signal are defined in step 602. Step 603 is a removal of a linear trend from the input signal y_all, and removal of distractions of high frequency by replacing them by averaging of the closest neighbours, usually 1000 or more neighbouring responses, assuming that there is no correlation between distractions and the response of the auditory system, the result is marked as y1_all. This means that the algorithm rejects measurements which have too big of artifacts. In step 604 an approximation of the signal y1_all is performed for a given sampling rate, such as 100 kHz, and outlier data is cut, and marking them as y2_all. With the exception of the first 10 measurements, for each measurement made the variance of the signal is calculated. If the variance of the signal determined is greater than double the arithmetic mean of the previous standard deviations, then the result is not included in the averaging signal. It was assumed that if a sample deviates from zero by more than four standard deviations of the signal, it is replaced with the average value of neighbouring samples that deviate from zero to less than 4 standard deviations. In order to reduce the effect of transient states, for signal analysis it is advisable to take the signal from the interval [1, 10 ms], wherein the first sample corresponds to the time t=1 ms, and the last one to the time t=10 ms. In step 605 a signal approximation with the usage of the gausso-like functions is carried out in such a way that the tracking error is as small as possible, which results in eliminating small artifacts. For this purpose resampling the signal is performed so that the number of the samples is 512. Resampling is made, by approximation, to the signal between sampling points using a linear function. At the end, low-pass filtering is performed by a digital filter in which cut-off frequency is equal to 0.5 value of the sampling frequency. In order to locate the wave in step 606, intervals of maximum values are determined with the use of the second derivative. In step 607, the width of peak points are determined by the usage of the points of second derivative. During step 608 the maximum values that exceed the noise too little are discarded. In step 609 maximum values which are too narrow are discarded. If for a given wave more than one group is formed, then in step 610 matrix of transfers D is created. In step 611 elements of maximum values are grouped by leads discarding those which protrude too much. In step 612 the probability of belonging to a given wave using a density function is calculated. In step 613, from a given wave, which belongs more than one group, the one with the biggest value of probability is chosen and the final shape of ABR waveform is determined. In the step 614 the procedure of wave detection is terminated.

To summarize, the signal recording and the signal processing for diagnosing the auditory start after connecting the electrodes to the head skin and near the auditory system. Then the data of bioelectric signals of brain activity or eye or muscle twitching: EEG or EOG or EMG or their combinations, are recorded, and the signal evoked by the auditory brainstem response signal (ABR) caused by acoustic stimulation of the auditory system, which is processed in graphic form in order to obtain information on the brain's response to acoustic stimulation, removing artifacts caused by the movement of electrodes or wires and muscle artifacts. The recording shows its course over time in a graph. The signal recording and the signal processing for diagnosing the auditory is characterized in that the first charge signal is bioelectrical activity of the brain or eye or muscle and is measured by the independent measuring channel of EEG or EOG or EMG or their combination from the electrodes connected on the head skin, which is converted from analog to digital, and after observing the optimal baseline ABR sleep phase signals, generated acoustic stimulation of the auditory system by acoustic stimulation circuit and activating signal generated by the system informing to start observing the optimal sleep phase ABR testing, which shall be automatic activation of an independent channel for ABR signal acquisition. Later it automatically activates independent ABR circuit signal acquisition and signal ABR is collected by electrodes placed in the vicinity of the auditory system and independent channel for ABR signal acquisition, which amplifies the amplifier to a voltage not higher than 500 mV, and is limited to a range of transfer from 0 Hz to 5000 Hz by means of filters, and then performs a signal conversion of ABR signal of independent channel of ABR signal acquisition from analog to digital and records ABR signal of independent channel for ABR signal acquisition in a configurable time interval measured from the acoustic stimulation, and ABR signal of independent channel for ABR signal acquisition are presented in graphical form on a screen or on paper.

ABR signal recording can be made available in a graphic form using the component for visualization and description of ABR signal. The computer program allows the user to record both the presentation of the original signal and the signal filtered by the algorithm. It also has the ability to compare two signals simultaneously through their presentation on the screen. Waveform of signals can be sorted and decomposed proportionally to the value of the stimulus intensity. The user can configure the workspace of the component. All tests can be displayed individually, adjacently or superimposed on one other. Each waveform can be rescaled independently on timeline and amplitude axis. Specific values of signal samples can be read by the user. Values for all of the signals are displayed, both original and filtered. Together with values, intervals which are in between characteristic points are presented, for example, intervals I-III. On every waveform 621-630 out of the set of 620 waveforms in FIG. 15, the user can enter characteristic points, such as wave I together with the other waves III and V. Waveforms 621-630 of ABR signals are waveforms of the patient who hears correctly in the presence of decreasing intensity of the acoustic signal. Each point can be described. The system can edit, delete and move points throughout the waveform. In addition, the component provides tools supporting the work over the examination, which are conventionally called ‘magnet’, ‘min/max’, ‘snapshot’ and ‘filtering by type’. The tool “magnet” facilitates taking bearings of characteristic points on the waveform; the tool “min/max” determines local minimum or maximum values in the specified area of the chart; tool “snapshot” provides precision applying the characteristic points, even with an accuracy of a single sample, and the last of these tools allows to fitter the visible characteristic points by type.

While the technical concept presented herein has been depicted, described, and has been defined with reference to particular preferred embodiments, such references and examples of implementation in the foregoing specification do not imply any limitation on the concept. It will, however, be evident that various modifications and changes may be made thereto without departing from the broader scope of the technical concept. The presented preferred embodiments are exemplary only, and are not exhaustive of the scope of the technical concept presented herein. Accordingly, the scope of protection is not limited to the preferred embodiments described in the specification, but is only limited by the claims that follow.

The invention claimed is:

1. A system for recording and processing a signal for diagnosing an auditory system comprising
an audio channel for acoustic stimulation of the auditory system and generating acoustic signals;
a system for measuring signals of bioelectric activity of brain (EEG signal) or eye (EOG signal) or muscle (EMG signal) or their combination and having
an independent measuring channel of EEG or EOG or EMG signals or their combination obtained from electrodes attached to a head skin of a person to be tested and comprising an analog-to-digital converter and a signal output for outputting a sleep phase signal informing about sleep phase;
a system informing about sleep phase occurrence and generating an activating signal when sleep phase occurs and having
a signal input for becoming the sleep phase signal from the signal output of the system for measuring signals of bioelectric activity, and
a signal output for outputting the activating signal;
a system for measuring an auditory brainstem response signal (ABR signal) evoked by acoustic stimulation and having
electrodes attached to the head skin near to the auditory system of the person to be tested,
an independent ABR channel for ABR signal acquisition connected to the electrodes of the system for measuring the ABR signal and having amplifiers amplifying the ABR signal, an analog-to-digital converter and an activation input;
a circuit for automatic activation of the independent ABR channel having an activating input communicating with the signal output of the system informing about sleep phase occurrence and an activation signal output communicating with the activation input of the independent ABR channel when the sleep phase occurs;
amplifiers and filters for amplifying the ABR signal up to 500 mV obtained from an independent channel for ABR signal acquisition and for reducing bandwidth using filters ranging from 0 Hz to 5000 Hz;
a recording system of the ABR signal of the independent channel for ABR signal acquisition in a configurable period of time measured from the acoustic stimulation;
a system for the analysis and processing of EEG or EOG or EMG signals or their combination and the ABR signal to obtain information on the brain's response to acoustic stimulation and to remove artifacts caused by the movement of the electrodes or cables and muscle artifacts; and
a system to present in graphic form the ABR signal of the independent ABR channel.

2. The system for recording and processing the signal for diagnosing the auditory system according to claim 1 wherein the independent measuring channel of EEG or EOG or EMG signals or their combination and the independent ABR channel for ABR signal acquisition are connected via a processor of the system and a communication interface of systems for external communication with a transmitting device to a unit which receives and processes data from measurements made by the independent measuring channel of EEG or EOG or EMG signals or their combination and the independent ABR channel for ABR signal acquisition.

3. A method for recording and processing signal for diagnosing an auditory system, the method comprising
attaching electrodes of a system for measuring signals of bioelectric activity to a head skin;
connecting electrodes of the system for measuring signals of bioelectric activity to an independent measuring channel of EEG or EOG or EMG signals or their combination;
attaching electrodes of a system for measuring an auditory brainstem response signal (ABR signal);
connecting the electrodes of the system for measuring the ABR signal to an independent ABR channel for ABR signal acquisition;
measuring EEG or EOG or EMG signals or their combination to detect sleep phase occurrence;
converting the EEG or EOG or EMG signals or their combination from an analog form to a digital form;
outputting a sleep phase signal informing about sleep phase to a system informing about sleep phase occurrence;
generating an activating signal when sleep phase occurs;
automatically activating the independent ABR channel for ABR signal acquisition;
generating acoustic stimulation signals for stimulation of the auditory system;
measuring the ABR signal collected through electrodes using the independent ABR channel;
amplifying the ABR signal to a voltage not higher than 500 mV and limiting ABR signal frequency to a range of 0 Hz to 5000 Hz by filters;
converting the ABR signal from an analog form to a digital form;
recording the ABR signal in a configurable period of time measured from time the acoustic stimulation signals are generated; and
presenting the ABR signal of the independent channel for ABR signal acquisition in graphic form on a screen or on paper.

4. The method for recording and processing the signal for diagnosing the auditory system according to claim 3 further comprising
sending the EEG or EOG or EMG signals or their combination and the ABR signal after converting to the digital form via a communication interface of external communication systems with a transmitting device to a unit which receives and processes data of the EEG or EOG or EMG signals or their combination and the ABR signal obtained from the system for measuring signals of bioelectric activity and the system for measuring the ABR signal.
